(19) United States
(12) Patent Application Publication
(10) Pub. No.: US 2004/0110199 A1
(43) Pub. Date: Jun. 10, 2004

(54) Microfluidic affinity system using polydimethylsiloxane and a surface modification process

(76) Inventors: Carlo D. Montemagno, Los Angeles, CA (US); Elsa I. Chang, Beaverton, OR (US)

Correspondence Address: JONES, TULLAR & COOPER, P.C., P.O. BOX 2266 EADS STATION, ARLINGTON, VA, 22202

(21) Appl. No.: 10/650,042

(22) Filed: Aug. 28, 2003

Related U.S. Application Data
(60) Provisional application No. 60/406,312, filed on Aug. 28, 2002.

(51) Int. Cl. B05D 3/00; C12Q 1/68; G01N 33/53; C12M 1/34

(52) U.S. Cl. 435/6; 435/7.1; 435/287.2; 427/2.11

(57) ABSTRACT

A microfluidic affinity system is designed to recognize, capture and separate target analytes from input solutions. This microfluidic affinity system employs fluidic channels fabricated by silicon-based lithography in a silicon substrate. The fluidic channels are patterned and replicated in a substrate, preferably polydimethylsiloxane, PDMS, by pattern transfer from a silicon wafer mold with reversed patterns fabricated by lithography. A novel three-step covalent binding method for surface modification employs the following steps to covalently immobilize an affinity ligand on the substrate: 1) a plasma treatment; 2) a silanization treatment; and 3) a crosslinking treatment.

### CROSS-REFERENCE TO RELATED APPLICATION

This application claims the benefit, under 35 U.S.C. 119(e), of U.S. Provisional Application No. 60/406,312 filed Aug. 28, 2002, the contents of which are incorporated herein by reference.

### BACKGROUND OF INVENTION

1. Field of the Invention

The present invention relates to a microfluidic affinity system, preferably a pathogen-detection system, and a method for producing the microfluidic affinity system by combining affinity technology and bioelectronics technology.

2. Description of Related Art

Cryptosporidium parvum is a waterborne parasite responsible for several serious outbreaks of cryptosporidiosis illness worldwide. In 1993, an outbreak of Cryptosporidium parvum occurred in Milwaukee, Wis. resulting in 400,000 infections and several deaths. This pathogen can be transmitted in an oocyst form through water and the oocyst is resistant to many environmental stresses and water treatment practices. The numbers of oocysts in environmental waters are very low. However, the minimum infectious amount of oocysts is small. Therefore, rapid, specific, and sensitive detection methods are needed to determine the prevalence of Cryptosporidium in environmental samples and the assessment of the potential risk to public health. Current detection methods rely on microscopic examination, immunofluorescent, acid-fast staining, and polymerase chain reaction, which lack assay sensitivity or are labor-intensive and time-consuming. It would be advantageous to develop a detection method with increased sensitivity for this and other health threatening pathogens.

Affinity chromatography is a process used for separating mixtures by using immobilized affinity ligands to interact with targeted substrates. An affinity chromatography device typically contains two parts: affinity ligands and a matrix support. Affinity ligands contain binding sites specific for target substrates. Therefore, the affinity ligand has the ability to capture target substrates. Typically, affinity ligands are firmly immobilized on a matrix support by covalent means. Common affinity ligands include immunoglobulin, enzyme inhibitors, various biospecific binders, metal ions, drugs, and receptor proteins. A matrix support is any insoluble material to which an affinity ligand can be attached. The insoluble matrix support is usually solid. Hundreds of natural and synthetic substrates have been employed as affinity matrices such as ground shrimp, grass pollen, agarose beads, polyacrylamide beads, Ultrogel AcA gel, and Azlactine beads. For example, the beads serve as a solid matrix support and affinity ligands are attached on the beads. The affinity chromatography techniques are most useful and powerful when applied to isolation and purification, capture and detection, selective removal, enzymatic catalysis and chemical modification.

It was reported that advances in the area of affinity chromatography and innovations in the emerging field of bioelectrical systems have been combined to produce new revolutionary detection systems. The affinity chromatography method is based on the specific affinity between immobilized receptors and their specific target analytes. By this specific interaction, target analytes can be captured and separated from mixture solution. Adapting these specific interactions to an electrical measurement system, novel detection systems can be created and be applied to such diverse fields as diagnostics, therapeutics, processes control, waste and environmental monitoring and kinetic analysis of the interaction of various biological substances. This technology merging offers a novel, new concept for a better pathogen-detection system. Bioelectronic detection systems have been studied previous. Taking advantage of the electrical properties inherent in certain types of biomolecules, an electronic sensor was constructed for counting and checking the amount and viability of Cryptosporidium parvum oocysts in water samples.

The development of a small-size affinity chromatography device with good capacity is key to the optimization of a pathogen-detection system. However, conventional affinity chromatography techniques employ a tall column containing solid beads on which the affinity ligands are immobilized. When the analtye-containing solution runs through the column, the target analytes can be captured by immobilized affinity ligands. The large size of the conventional affinity chromatography device makes it unsuitable for portable and in-situ application. In addition, the varieties of bead size and pore configuration are limited by commercial availability.

The physical and chemical durability of a substrate used as the matrix support can affect the performance of affinity chromatography-based separation. When choosing a substrate for a microfluidic affinity system, the substrate properties should be considered. Some synthetic substrates have been developed to give better affinity chromatography-based separation. The flow characteristics processed by the substrate play a very important role in the performance of an affinity chromatography system. The flow characteristics of a substrate depend largely on the substrate particle size, the pore size and the pore configuration. A narrower particle size produces a more efficient column capacity because of less frequent column channeling and greater concentration of final eluted product. In addition, narrower particle sizes can result in a reduced void volume and a larger flow rate. A suitable pore size and pore configuration within a particle can help the analyte to successfully diffuse into the binding sites of affinity ligands in the pores.

The use of silicon and glass as the substrate of the affinity chromatography device presents a variety of problems, including product throughput and cost. In addition, the channel sealing processes for silicon and glass are complicated and time consuming. Further-more, silicon and glass materials are fragile and too expensive for disposal.

Silicon-based lithography, the process of pattern transfer to produce devices on micrometer and nanometer scales, offers an alternative solution resulting in the development of a fine network for the microfluidic affinity system. Silicon-based lithography is the driving technology to the reduction of critical dimensions, which at this time can resolve features on the order of 100 Å with electron beam techniques.

A number of researchers have worked on making microfluidic devices from PDMS polymers replicated from silicon masters instead of making the devices directly in the silicon or glass for throughput and cost reduction. The PDMS microfluidic systems have been applied in DNA separation, microvolume polymerase chain reaction, enzyme assay, and immunoassay. However, a big challenge is presented in the immobilization of affinity ligands on a PDMS channel wall. The PDMS elastomer surface is hydrophobic and does not have derivatizable functional groups for subsequent modification and attachment of an affinity ligand. The surface properties of the PDMS elastomer make immobilization of receptor ligands on PDMS surfaces difficult. Therefore, the affinity ligand-PDMS-surface interactions are an important aspect in the development of a method for immobilization of affinity ligands on PDMS substrates.

Plasma treatment is a technique used in modification of silicone elastomer surfaces and it has been used to increase the wettability of PDMS for improved compatibility to other materials, e.g. in biomedical applications and printing technology. Many researchers have reported hydrophobicity loss of silicone elastomers when treated with plasma discharge. One researcher reported that a microfluidic system was designed in PDMS elastomer to separate amino acids, charged proteins and DNA fragments in aqueous solutions. A silicon master with a network of microfluidic channels was created by photolithography and PDMS was caste against the master to yield a polymeric replica. The channels were sealed by conformal contact of two plasma oxidized PDMS surfaces. Oxidized PDMS also seals irreversibly to other materials used in microfluidic systems, such as glass, silicon, silicon oxide, and oxidized polystyrene. The mechanism of reversible binding between two oxidized PDMS is that plasma discharge converts —OSi(CH₃)₂O— groups at PDMS surfaces to —OₙSi(OH)₄₋ₙ. When two oxidized PDMS are brought to conformal contact, condensation reaction between two silanol groups on two contact surfaces results in covalent siloxane bonds (Si—O—Si). Yet another researcher described a procedure for making topologically complex three-dimensional microfluidic channel systems in PDMS. Basic fabrication processes for PDMS also used the replication of a master and the condensation binding between two oxidized PDMS. A new “membrane sandwich” method was developed to stack and seal more than one PDMS slab for complicated geometries of 3D microfluidic systems.

### BRIEF SUMMARY OF THE INVENTION

The present invention is a method for producing a microfluidic affinity system, preferably for pathogen detection, through the combination of affinity chromatography technology and bioelectronics technology. In order to provide a completely functional detection system, a small, portable affinity system has been developed for recognition and capture of target biomolecules in solution. This small, portable affinity system can then be suitably combined to an electronic measurement system for in-situ quantification and viability-measurement of target biomolecules.

Instead of using a conventional bead and column system, a microfluidic affinity system is constructed by fabricating fluidic channels using silicon-based lithographic techniques on a silicon or glass substrate as a matrix support. Microscale or nanoscale fluidic channels can be fabricated this way. Therefore, specific channel size and morphology for a microfluidic affinity system can be designed for any type of analytes to optimize the system performance.

Polydimethylsiloxane, PDMS, a silicone material, is used as a preferred substrate for the matrix support of the microfluidic affinity system. PDMS is less expensive and less fragile then glass and silicon. The processes used to create the fluidic channels on a PDMS elastomer are based on replication and are more efficient and less expensive. Standard silicon-based lithography is used to controllably generate a master pattern onto a silicon wafer. This master wafer, with reversed fluidic channel patterns, can then be used repeatedly as a mold for a PDMS cast for pattern transfer to a PDMS substrate.

The present invention uses a novel surface-modification method for protein covalent binding on a surface of the PDMS substrate, herein referred to as the three-step covalent binding method. A plasma treatment is employed to introduce derivatizable functional groups on the surface of the PDMS substrate for subsequent modification and attachment of affinity ligands. The surface of the PDMS substrate is then subjected to a silanization treatment and a crosslinking treatment. The novel three-step covalent binding method displays the desired properties for larger amounts of affinity ligand immobilization and higher antigen-capturing activities on the surface of the PDMS substrate. When this novel three-step covalent binding method is applied to the substrate, preferably PDMS, used as the matrix support in the microfluidic affinity system, the system shows the ability to capture target analytes from input samples. The system can also be regenerated by releasing the captured analytes using acidic rinsing.

### BRIEF DESCRIPTION OF THE SEVERAL VIEWS OF THE DRAWINGS

The features and advantages of the present invention will become apparent from the following detailed description of a preferred embodiment thereof, taken in conjunction with the accompanying drawings, in which:

FIG. 1 is a three-dimension structure of PDMS polymer;

FIG. 2 is the chemical structure of PDMS polymer;

FIG. 3 is a representation of direct ELISA;

FIG. 4 is a representation of sandwich ELISA;

FIG. 5 is a graphical representation of the relation between chemiluminescent intensity and anti-chicken IgG-P concentration;

FIG. 6 is a graphical representation of immobilized chicken IgG activity, or surface activity, on PDMS as a function of chicken IgG loading concentration;

FIG. 7 is a graphical representation of the total surface activities and the activities from non-specific adsorption at different pH values;

FIG. 8 is a graph of the ionic strength effect on chicken IgG immobilization on PDMS;

FIG. 9 is a representation of the PDMS elastomer surface modification, antibody immobilization, and antigen capture;

FIG. 10 is a graph of the surface activities of attached chicken IgG on completely modified PDMS, non-modified PDMS, and incompletely modified PDMS;

FIG. 11 is a graphical comparison of surface activities resulted from different chicken IgG loading concentrations;

FIG. 12 is a graph of the stability of immobilized chicken IgG on PDMS by novel surface modification over 21 storage days;

FIG. 13 shows the original microfluidic channel pattern drawn in SYMBAD to simulate porous media;

FIG. 14 is a schematic of making fluidic channels in a PDMS elastomers;

FIG. 15 is a picture of fluorescent dye pumped through the fluidic channels at the flow rate of 6 ml/hr;

FIG. 16 is a graph of a standard curve of ant-chicken IgG-P concentration versus intensity;

FIG. 17 is a graph of anti-chicken IGG-P concentrations of output samples from a microfluidic affinity systems;

FIG. 18 is a graphical comparison of captured anti-chicken IgG-P amounts when applied with high and low input anti-chicken IgG concentrations;

FIG. 19 is a graph of the standard curve of protein concentration versus A₂₈₀;

FIG. 20 is a graph of the A₂₈₀ of output fractions after rinsing the channels with glycine-HCl (pH2.3) solution;

FIG. 21 is a graphical comparison of output anti-chicken IgG-peroxidase (antigen) concentrations over 1^{st} to 3^{rd} use of the microfluidic affinity system; and

FIG. 22 is a graphical comparison of anti-chicken IgG-peroxidase capturing amount at 1^{st} to 3^{rd} use of the microfluidic affinity system.

### DETAILED DESCRIPTION OF THE INVENTION

A new microfluidic affinity system is designed to recognize, capture and separate target analytes from input solutions. The microfluidic affinity system includes a silicone material as a substrate for a matrix support for the immobilization of affinity ligands. Preferably, fluidic channels are fabricated in the silicone material using silicon-based lithography. The fluidic channels are patterned and replicated in a silicone substrate, preferably polydimethylsiloxane, PDMS, by pattern transfer from a silicon wafer mold with reversed patterns fabricated by lithography. Therefore, fluidic channels in a silicone substrate, preferably PDMS, can be produced with precision and controlled rapidly and economically.

Antibodies and proteins are used as preferred affinity ligands to be immobilized on the substrate, preferably PDMS, in the microfluidic affinity system. The substrate, preferably PDMS, is subjected to surface modification in order for the affinity ligands to immobilize on the substrate. A novel three-step covalent binding method for surface modification employs the following steps: 1) a plasma treatment; 2) a silanization treatment; and 3) a crosslinking treatment. This three-step covalent binding method is used to covalently immobilize affinity ligands on the substrate, preferably PDMS.

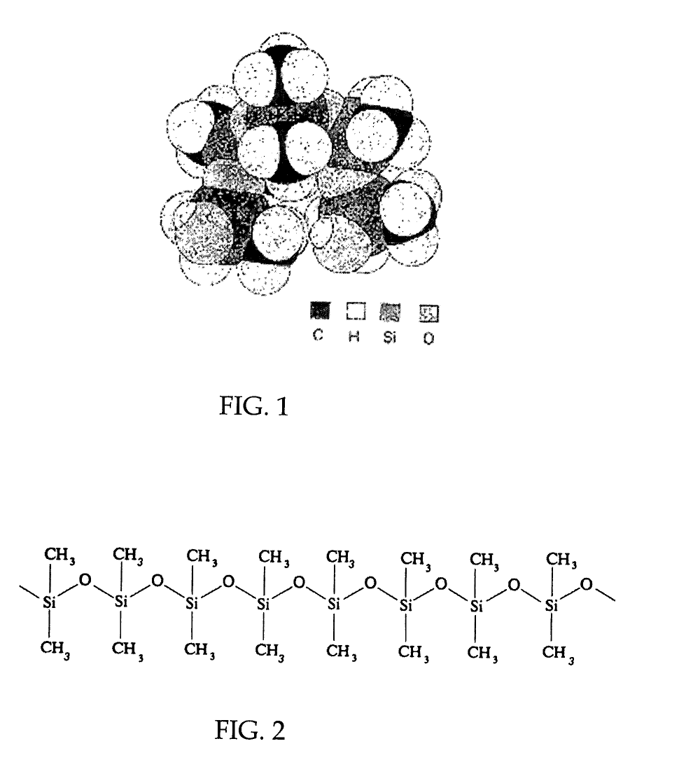

Polydimethylsiloxane, PDMS, a silicone material, is the preferred substrate used as the matrix support for affinity ligand immobilization in the microfluidic affinity system. PDMS consists of an inorganic backbone of alternating silicone and oxygen atoms, see FIGS. 1 and 2. Methyl groups are attached to the silicon atoms, forming the repeat unit in the polymer. Linear PDMS may be manufactured from dimethyldichlorosilane, which is produced by the reaction of powdered silicon with methyl chloride, catalyzed by copper, as shown below in Reaction 1. This reaction gives several chlorosilane intermediates. Dimethyldichlorosilane is the main product and it is separated from other chlorosilanes by distillation.

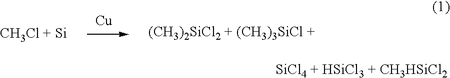

The chlorosilane monomers are separated by fractional distillation and are then hydrolyzed to yield cyclic and linear siloxane polymers as shown in Reaction 2.

(CH₃)₂SiCl₂+H₂O→HO[(CH₃)₂SiO]ₓ+[(CH₃)₂SiO]_{3,4,5}+HCl (2)

Higher molar mass polymers are then produced by ring-opening polymerization or polycondensation.

High silicone gum with molecular weight between 300,000 to 700,000 g/mol is used. Crosslinking of the polymer chains takes place through decomposition of peroxides at temperatures greater than 100° C. The peroxides decompose into free radicals, which react with unsaturated bonds or even with methyl groups by abstraction of hydrogen atoms.

Two different crosslinking methods are used for room temperature vulcanized PDMS elastomer. One method uses a condensation reaction of silanol group, Si—OH, to form siloxane bonds by liberation of water, see Reaction 3. The reaction involves water and is catalyzed by acid and base.

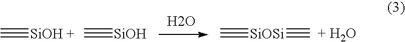

The second reaction is hydrosilylation. This reaction involves the addition of silicon-hydrogen, SiH, to an unsaturated carbon bond, usually a vinyl group, —CH═CH2, catalyzed by a noble metal, e.g. Platinum, see Reaction 4.

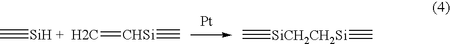

The second reaction is very specific and crosslink density can be controlled very accurately by this method.

Since the inorganic siloxane backbone of PDMS is composed of more than one atom, the electronegativity difference between the atoms causes the backbones to be partially polar or ionic and partially covalent. It also causes such bonds to be more susceptible to nucleophic or electrophilic attachment than wholly covalent linkages. The Pauling electronegativity difference of 1.7 between silicon and oxygen confers a 41% polar or ionic character on the siloxane bond. Its consequent sensitivity to hydrolysis at extremes of pH is the most significant different between silicones and other organic polymers, see Equation 5.

—Si—O—Si—+H2O ⇄2—SiOH (5)

Hydrolytic splitting of the skeletal siloxane polymer bonds becomes appreciable at pH values of the aqueous substrate smaller than 2.5 and greater then 11.0.

Plasma treatment is a technique used in modification of silicone elastomer surfaces and it has been used to increase the wettability of PDMS for improved compatibility to other materials, e.g. in biomedical applications and printing technology. Many researchers have reported hydrophobicity loss of silicone elastomers when treated with plasma discharge. It has been shown that the complexity of plasma exposure is partly because the polymer is subjected simultaneously to a mixture of energetic species and radiation, e.g. electrons, ions, UV, and ozone. A large number of reactions take place and the reaction products make the surface of PDMS hydrophilic. The main effects of plasma treatment on PDMS is summarized as follows: (a) the formation of a glassy SiOₓ surface layer; (b) the increase in oxygen content in the surface by the formation of SiOₓ, hydroxyl and carbonyl groups; (c) degradation of the network structure resulting in the formation of mainly low molar mass cyclic and medium to high molar mass linear PDMS.

Hydrophobicity recovery normally occurs gradually over time after the cessation of plasma discharge. Many mechanisms have been proposed for hydrophobicity recovery of PDMS elastomer after exposed to plasma, possibilities including:

1. Migration of low molar mass species from the bulk to the surface.

2. Reorientation of polar groups at the surface into the bulk.

3. Condensation of the silanol (Si—OH) groups at the surface.

4. External contamination of the surface.

5. Change of surface roughness.

6. Loss of volatile species to the atmosphere.

Of these proposed mechanisms, there is a consensus that migration of low molar mass of PDMS to the surface is the dominant mechanism. The oxidation to a SiOx surface layer retards the migration of low molar mass PDMS. However, cracking of the SiOx layer enhances the rate of hydrophobic recovery.

The “phase-change from fluid to elastomer after cure” property of PDMS makes it an ideal material for the microfluidic affinity system. The fabrication processes for construction of the microfluidic affinity system are based on PDMS elastomer replication of masters that have been made by other means, often by lithographic processes on silicon or glass. One master can be used repeatedly to produce many PDMS replications rapidly. Beside the cure/replication steps, the bonding step is also very important in the PDMS elastomer fabrication processes. In the binding step, a cover layer is bound to close the fluidic channels. It has been reported that plasma oxidized PDMS elastomers can immediately form a reversible binding at room temperature when pressed together.

Various sensor technologies require tightly bound affinity ligands, such as proteins, on the substrate used as the matrix support. Different attachment methods will result in different characteristics of immobilized affinity ligands. There are generally two different types of attachments on the surface of a substrate: covalent binding and non-covalent adsorption. Each attachment type and different surface modification methods for attachment will be discussed below.

Proteins, preferred affinity ligands, can be attached to a surface of a substrate by the covalent bond linkages between proteins and the surface. Hereinafter, it will be understood that the use of proteins as the affinity ligand is merely an example used for simplicity. This bond is formed by the virtue of sharing valence electrons between atoms. Covalent bonding is essentially irreversible and thus more stable for sensor application of protein attachment. The formation of covalent bonds between proteins and the surface depends on the derivatizable functional groups on both proteins and the surface. Surface modification and protein activation procedures are usually necessary for covalent protein attachment on the surface.

Non-covalent adsorption is based on the mechanisms of electrostatic reactions, such as ionic bonds and Van der Waals forces, and hydrophobic interaction between protein molecules and the surface. An ionic bond is an electrostatic bond that forms when a protein and the surface are oppositely charged. This binding is caused by the Coulomb attraction between opposite charges. The Van der Waals bond is a very general term for several types of weak bonds, e.g. dipole-dipole force, dipole-induced force, and dispersion force between the protein molecules and surface molecules. Hydrophobic interaction does not involve electrostatic forces. Non-polar molecules are driven together in water by entropically favorable reactions. The entropy increases when hydrophobic reactions occur in the solution. As the groups on the solid surface are non-polar, the non-polar part of the protein molecule can be driven to the non-polar surface by hydrophobic interactions.

Different attachments involve different types of protein-solid-surface interaction forces. The protein-solid-surface interaction is an important parameter determining the amount and stability of immobilized proteins. Attachment of proteins on solid surfaces can be achieved by adsorption. The resulting surface loading of proteins in some cases is the same or even higher than that of a covalent attachment. However, the proteins immobilized by adsorption suffer partial denaturation and tend to leach or wash off the surface.

By way of example, the kinetics and stability of immobilized immunoglobin G, IgG, on silica surfaces has been analyzed. As the conditions for covalent attachment of IgG to the surfaces are fulfilled, the IgG showed high affinity, and the immobilized amount of IgG showed a fast saturation. Changes of ionic strength in the IgG solution during the attachment step showed no significant influence on the immobilization kinetics and the saturated amount of attached IgG on silica surfaces.

Covalent binding of IgG also shows high stability towards changes in the surrounding medium such as lowering the pH or surface tension, incorporation of detergents and increase in ionic strength. In contrast, non-covalent attachment is reversible and hence has less stability towards changes in environment. Electrostatic attachment and hydrophobic attachment of proteins are both non-covalent attachments. However, they reveal different levels of stability when exposed to different medium changes. For example, when washed with detergent the IgG attached by hydrophobic interaction could be significantly eluted, whereas IgG adsorbed on surfaces by electrostatic interaction was not markedly influenced by this treatment.

It is also reported that lowering of surface tension by ethylene glycol in combination with increased ionic strength eluted significant amount of proteins that are attached on surfaces by electrostatic interaction. Furthermore, lowering the pH elutes some proteins from surfaces for both electrostatic and hydrophobic attachments.

Proteins, the preferred affinity ligand, contain several types of reactive groups that can be used to form covalent bonds to some groups on solid substrates. The most frequently used protein active groups are the amino groups, followed by sulfyhydryl, carboxy and aromatic groups. The preferred functional group on the surface of the substrate for covalent protein binding can be an aliphatic amine, sulfhydryl, aromatic amine, or epoxy group. However, those functional groups are usually not present on the surface of silicon and glass. Therefore, surface modification procedures are required to introduce the suitable functional groups on the glass and silicon surfaces for protein binding. A silanization treatment is one of the most often used methods for introducing desirable functional groups on the surface of silicon and glass. Silanization introduces a thin silane layer on the surface and the suitable functional groups can be introduced this way. Preferred silanes include aminosilane, sulfhydrylsilane, and epoxysilane. Silanization treatments are of two types: aqueous and organic. Aqueous silanization offers the advantage of more uniform coverage and an apparently thinner and uniform silane layer on the substrate. Organic silanization produces a thicker, uneven, more loosely bound but higher capacity coating.

The hydroxyl group plays an essential role for silanization treatment on silicon and glass surfaces and subsequently enhances the surface modification and protein binding. The hydroxyl group can be activated in the form of silanol, Si—OH, on silicon and glass surface after treated with aggressive acid wash. Through this silanol group, the silane layer can be chemically linked to the silicon and glass surface. Therefore, the presence of hydroxyl groups determines if a surface is derivatizable and hence applicable for protein coupling.

After the silanization treatment, the next step is to link the active groups on proteins to the functional groups introduced by silanization on the glass or silicon surface, herein referred to as a crosslinking treatment. The usage of a linker is involved here. The linker may have the same reactive function on each end, homobifunctional, or the two ends may have different functional groups, heterobifunctional. Two examples of commonly used homobifunctional and heterobifuctional linkers are glutaraldehyde and N-γ-maleimidobutyryloxy succinimide ester (GMBS) respectively. In either case, the free group is capable of reacting with the protein.

There are two preferred protein immobilization methods for silicon surfaces that have been subjected to a plasma treatment. The first immobilization method is based upon subjecting the surface to a silanization treatment using aminopropyltrimethoxysilane (APTS) derivatization followed by a crosslinking treatment using glutaraldehyde. The second method subjects the surface to a silanization treatment using a mercaptopropyltrimethoxysilane (MPTS) derivazation followed a crosslinking treatment using N-γ-maleimidobutyryloxy succinimide ester (GMBS). In the APTS/glutaraldehyde method, the triethoxysilanes, APTS, are known to polymerize in aqueous solutions leading to multilayer deposition on surfaces. Also, the linker used in this method, glutaraldehyde, also has self-polymerization. The multilayer of polymerized silane and linker result in polymerized multilayerd protein films. The MPTS/GMBS immobilization is not subject to either of these issues and in fact, was specifically tailored to provide monolayer protein coverage.

Previous research has investigated the effectiveness of both immobilization methods by the measurement of the amount of protein immobilized to silicon surfaces as well as the total activity of the proteins. Both chemistries resulted in good yields of immobilized enzyme and both appeared to be sufficiently mild to preserve half of the enzyme specific activity. However, the APTS/glutaraldehyde scheme performed better than the MPTS/GMBS immobilization in terms of enzyme load and total activity. The higher enzyme load and activity contributed to the polymerization of APTS and glutaraldehyde that provides more binding sites for protein attachment. APTS/glutaraldehyde was also simpler, cheaper and the more environmentally benign of the two methods, factors important in making mass-produced, inexpensive portable biosensors.

Immobilized affinity ligands can be made into powerful immunoaffinity supports that have the capability of binding and purifying almost any biological molecule. Antibodies are the most widely used affinity ligands in biosensor applications, and the immobilization of antibodies on solid surfaces became an important technology. For better antibody attachment, the effectiveness of an immobilization method needs to be judged. The measurements of the amount of antibodies immobilized to a surface as well as the activity provide good indicators for judging the effectiveness of binding the affinity ligands to a substrate. Although the following discussion focuses on the use of antibodies as the affinity ligand, it should be understood that this discussion does not limit the type of affinity ligand used in the microfluidic affinity system.

Measuring the total radioactivity of radioisotope labeled antibody is the most direct way to generate the calibration curve and quantify the amount of surface attached antibodies. Antibodies are first labeled with ¹²⁵I and then immobilized on the surface. The amount of surface-bound protein is determined using a scintillation counter, or gamma counter. Another method, Ellipsometry, measures the change in polarization of a light beam on the refection from a surface. This has been used by a number of workers to study the adsorption of proteins on solid surfaces. However, the radiolabelled antibodies were still required together with ellipsometry to calibrate the amount the immobilized antibodies.

The activity of immobilized antibodies can indicate not only the quantity but also the quality of antibody immobilization. Some attachment can result in high antibody loading but the immobilized antibodies lose most of the activity because the access of the active sites of antibodies are blocked due to bad binding orientation. Enzyme linked immunosorbent assay; ELISA, was reported to effectively determine the immobilized antibody activity. ELISA exploits the use of an enzyme attached to one of the reagents utilized in the test. Subsequent addition of relevant enzyme substrates/chromogens causes a color change; the results can be read both by eye and quantified using spectrophotometers. Two types of ELISA for the measurement of immobilized antibody activity are described below.

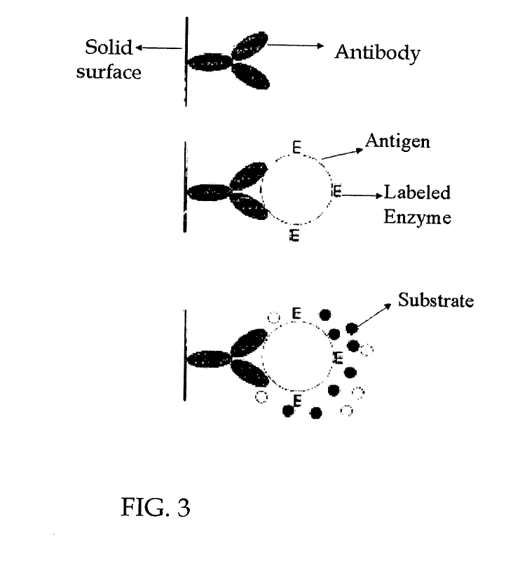

Direct-labeled antigen ELISA is shown in FIG. 3. The antigens labeled with enzymes are added to an antibody-attached surface and can be captured by antibodies immobilized on the substrate. The following washing step washes away uncaptured antigens. Relevant enzyme substrates are then added to react with the enzyme label and cause a color change. The color change is proportional to antibody activity, or antigen capture activity.

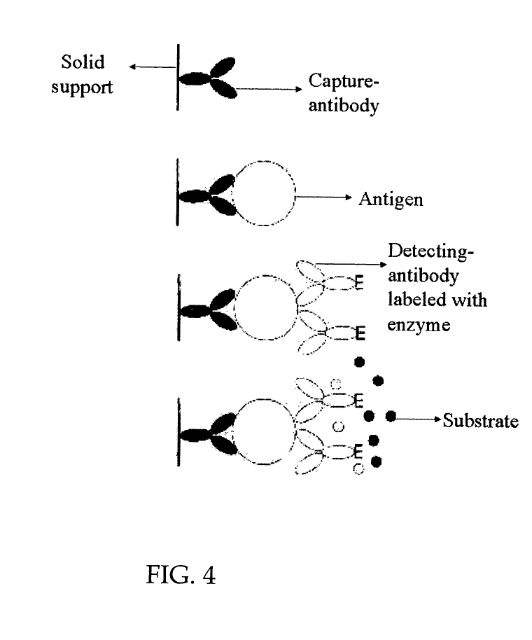

Sandwich ELISA is shown in FIG. 4. This method is used when an antigen is difficult to label with an enzyme. Un-labeled antigens are added and captured by antibodies attached to the substrate. The wash step washes away un-captured antigens. Enzyme-labeled antibodies are introduced here to react with captured antigens. The enzyme substrate then reacts with enzymes labeled on antibodies. Again, the color change can reflect the activity of antibodies attached on the surface of the substrate.

### Experimental Data

The first step of the experiments is the vulcanization of a PDMS elastomer. This step is for the preparation of PDMS elastomer slides as the substrate for the study of antibody/PDMS surface interactions. The experiment is categorized into two main parts.

The first part covers the investigation and analysis of antibody/PDMS surface interactions. Antibody/PDMS surface interactions involve both adsorption and covalent binding processes. Both interaction types are studied and compared. The objective is to find a better protein immobilization method with higher surface activity and stability.

The second part covers the construction of a microfluidic affinity system in a PDMS elastomer. The better antibody immobilization method for PDMS surfaces obtained from the first part of the experiment was used to attach antibodies for the microfluidic affinity system. The analyte-capturing efficiency of this microfluidic system was analyzed. In addition, the methods for regeneration of this microfluidic affinity system were investigated.

PDMS Vulcanization

The PDMS is in liquid phase before cure and is in the form of an elastomer after cure. PDMS elastomer slabs were prepared by mixing, deaerating, and curing steps. The resulting PDMS casts were used as the matrix support, or substrate, for the protein/solid-surface interaction experiment.

Mixing

Two components of room temperature vulcanization of PDMS rubber (RTV 615A&B, General Electric, Waterford, N.Y.) were placed with a 10: 1 mass ratio (RTV615 A: RTV615 B) in an evacuated flask 4-5 times larger than the volume of PDMS rubber to be used. The two components were stirred with a magnetic stir-bar.

Deaerating

To eliminate voids in the cured PDMS elastomer, the air entrapped during mixing should be removed. The mixed PDMS rubber components were exposed to a vacuum using a pump to displace the air from the mixing flask for approximately 1 hour.

Curing

The degassed PDMS rubber liquid was poured over a polystyrene Petri dish and cured in an oven at 65° C. for approximately 4 hours. The cured PDMS elastomer slab was cut into squares (0.5 mm×0.5 mm×0.2 mm) using an Exacto knife and peeled from the dish.

Investigation of Antibody/PDMS Surface Interactions: Adsorption

There are many methods to immobilize antibodies on surfaces. Among the immobilization methods, passive adsorption is the easiest one. It has been used very often for protein attachment on polystyrene surfaces for immunoassay. The objective of this experiment is to investigate the effectiveness of the adsorption method for antibody immobilization on PDMS surfaces. Differing antibody concentrations, pH, and ionic strength conditions of the absorption-based immobilization process were compared and analyzed to find the optimal conditions to control and increase antibody immobilization on PDMS. Analysis of pH and ionic strength effects on antibody immobilization can also help in the proposal of possible interaction forces that cause the adsorption of IgG on PDMS surfaces. Antibody concentration effect, pH effect, and ionic strength effect on antibody immobilization were investigated and the experimental methods and results are described below.

Investigation of the Relation between Loaded Antibody Concentration and Immobilized Antibody Amount on PDMS Surface

The antibody used in this experiment is chicken immunoglobulin G, IgG. The chicken IgG solutions with concentrations from low to high were incubated on PDMS slides, and the adsorbed chicken IgG on PDMS surfaces were tested for their analyte-capturing activity, also referred to herein as surface activity. The purpose of this experiment is to obtain the relation of concentrations of loaded chicken IgG and the resulting activity of adsorbed chicken IgG on PDMS; the adsorption saturation point of chicken IgG on PDMS was investigated as well.

The analyte for chicken IgG used here is anti-chicken-Immunoglobulin-G antibody, which is conjugated with an enzyme, peroxidase, referred to herein as anti-chicken IgG-P. When these enzyme-analyte complexes are added to a PDMS surface with immobilized antibodies, the analyte-enzyme complexes will be captured by antibodies with activities. Then, the enzymes conjugated on captured analytes can react with subsequently added compounds and form chemiluminescent products that can be detected by a spectrophotometer. The chemiluminescent intensity is proportional to the captured analyte-enzyme complex and, thus, is proportional to the immobilized antibody activity. This surface activity measurement is called the ELISA method that was discussed previously.

The chemiluminescent intensity readings at boundary ranges might not be proportional to the surface activity because of spectrophotometer resolution or enzyme-substrate reaction limitation. Therefore, the relation between anti-chicken IgG-P and its chemiluminescent product intensity was investigated to find the non-linear ranges.

Methods

a. Chicken IgG Adsorption

Chicken IgG (I4881, Sigma-Aldrich, Inc., Saint Louis, MI) in phosphate buffer saline solutions, referred to herein as PBS/pH 7.4, was prepared in a series of concentrations (0.05, 1, 10, 40, 100, 200 μg/ml). One PDMS elastomer slide in the dimension of 5 mm×5 mm×2 mm was incubated in 1 ml chicken IgG solution for each concentration at 37° C. for 3 hours. After incubation, the slides were washed with PBS/pH 7.4 containing 0.05% (v/v) Tween 20 using a wash bottle. Each side of a PDMS slide has been rinsed for 15 seconds continuously.

b. Non-Specific Site Blocking

PDMS slides with adsorbed chicken IgG were incubated in 1 ml PBS/pH7.4 containing 1% (w/w) bovine serum albumin, hereinafter referred to as BSA, in Microfuge tubes. This procedure is to block the non-specific binding sites on antigen immobilized PDMS surfaces. The whole blocking step was performed at 37° C. for 1 hour. The blocked PDMS slide was then washed with PBS/pH7.4 containing 0.05% Tween 20 using a wash bottle. Each side of the PDMS slide was rinsed for 15 seconds continuously.

c. Analysis of Chemiluminescent Intensity as a Function of Anti-Chicken IgG-P Concentration

Anti-chicken IgG-P in PBS/pH7.4 was prepared in a series of concentrations from 0.2 to 6.67 μg/ml. 50 ml of each concentration was added to 0.5 ml of chemiluminescent substrate solution (SuperSigal ELISA Pico Chemiluminescent Substrate, Pierce, Rockford, Ill.). Each sample was agitated gently by shaking for 15 seconds. Immediately, each sample was placed in the spectrophotometer to detect the intensity of chemiluminescent.

d. Measurement of Activity of Immobilized Chicken IgG on PDMS Slides

Each blocked and washed PDMS slide with adsorbed chicken IgG was incubated in 1 ml, 4 μg/ml anti-chicken IgG-P (A9046, Sigma-Aldrich, Inc., Saint Louis, Mo.) solution in a Microfuge tube at 37° C. for 1 hour. Each PDMS slide was washed again as in the previously described wash step. Each PDMS slide was then soaked in 0.5 ml, undiluted peroxidase substrate solution (SuperSigal ELISA Pico Chemiluminescent Substrate, Pierce, Rockford, Ill.) in a 1.5 ml polystyrene cuvette. Each sample was agitated gently by shaking for 15 seconds to increase the contact between fixed peroxidase on the PDMS surface and peroxidase in solution. Immediately, each sample was placed in spectrophotometer to detect the intensity of chemiluminescence that is the product of peroxidase-substrate reactions. The total activity of immobilized chicken IgG was defined as (chemiluminescence intensity)/(time) (total surface area).

Results

Analysis of Chemiluminescent Intensity as a Function of Anti-Chicken IgG-P Concentration

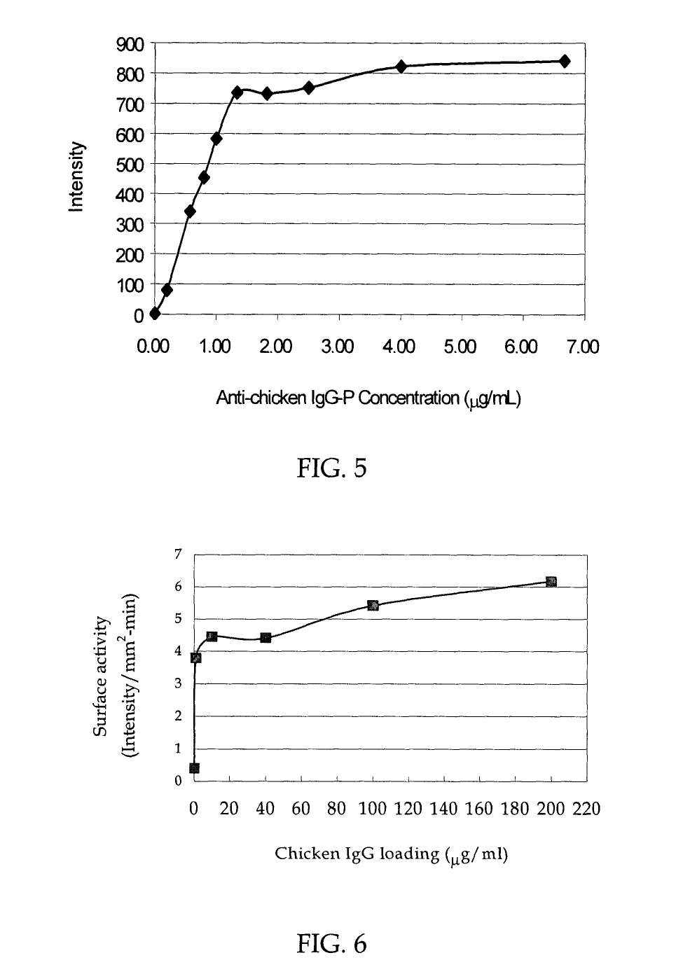

The chemiluminescent intensity as the function of anti-chicken IgG-P concentration is shown in FIG. 5. The intensity readings from 0 to 750 units are proportional to the anti-chicken IgG-P concentrations, as shown on the linear part of the curve. The intensity readings exceeding 750 units non-linear parts, not proportional to the corresponding anti-chicken IgG-P concentrations. Therefore, for all the immobilized chicken IgG activity measurements herein, the measured intensities will be controlled within this range, 0-750 units, so the measured intensities can reflect actual surface activities.

Measurement of Activity of Immobilized Chicken IgG on PDMS Slides (Surface Activity)

The activity of immobilized chicken IgG on PDMS, referred to herein as the surface activity, as a function of chicken IgG loading concentration at neutral pH is illustrated in FIG. 6. The surface activity increases sharply as the chicken IgG loading concentration increases from 0.05 to 10 μg/ml. From 10 to 100 mg/ml of loading chicken IgG concentration, the surface activity increases gradually. As the loading concentration exceeds 100 mg/ml, the surface activity is close to saturation and increases very slowly with the increasing chicken IgG loading concentration. Therefore, the chicken IgG loading concentrations below 100 μg/ml are more effective for chicken IgG adsorption and activity. The approximate saturation surface activity by the adsorption method at neutral pH environment is 5.4 Intensity/mm²-min. This result will be compared with that of covalent attachment method discussed herein.

Influence of pH on Immobilized Antibody Amount and Activity

Different pH might affect the protein and PDMS surface charges thereby causing different adsorbed antibody amounts and activities on PDMS surface. In this experiment, PDMS slides were incubated in chicken IgG solutions with different pH values. The resulting PDMS slides with adsorbed chicken IgG were neutralized and tested for their analyte-capturing activities, or surface activities, to find the best pH point for the optimization of chicken IgG adsorption. The activity mentioned above is defined as total surface activity.

Some analytes, such as anti-chicken IgG-P, are adsorbed on antibody-immobilized PDMS surfaces, instead of being captured by immobilized antibodies, such as chicken IgG. This non-specific adsorption of analytes was reduced by blocking non-specific sites with BSA for PDMS surfaces on which antibodies were immobilized by adsorption method. The remaining non-specific adsorption of analytes after the blocking step was investigated in this experiment. First, antibodies from any host other than chicken, such as sheep IgG, as used herein, were attached on PDMS surfaces by adsorption method. Second, anti-chicken IgG-P analytes were added to sheep IgG attached PDMS surfaces. Third, the surface activities were measured which are definitely attributed from non-specific adsorbed anti-chicken IgG-P. The activity described here is called non-specific activity.

Methods

10 μg/ml chicken IgG solutions were prepared in the following buffers with different pH values.

1. 0.1M, pH4.3 phosphate buffer

2. 0.1M, pH5.3 phosphate buffer

3. 0.1 M, pH6 phosphate buffer

4. 0.1 M, pH 7 phosphate buffer

5. 0.1M, pH8 phosphate buffer

6. 0.05M, pH9.6 carbonate-bicarbonate buffer

7. 0.05M, pH10.6 carbonate-bicarbonate buffer

8. 0.05M, pH 11.3 carbonate-bicarbonate buffer

PDMS slides were incubated in 1 ml of each IgG containing buffer, blocked with BSA and washed with PBS-Tween 20 according to the procedures described herein. Activity of immobilized chicken IgG at each pH control was measured and was defined as total surface activity. One set of the control PDMS slides was prepared to investigate nonspecific adsorption of anti-chicken IgG-P and the non-specific activities were measured as well. For this control set, all the procedures were the same as above, except that chicken IgG solutions were replaced by 10 μg/ml polycolonal sheep IgG (18265, Sigma-Aldrich, Inc., Saint Louis, Mo.) solutions.

Results

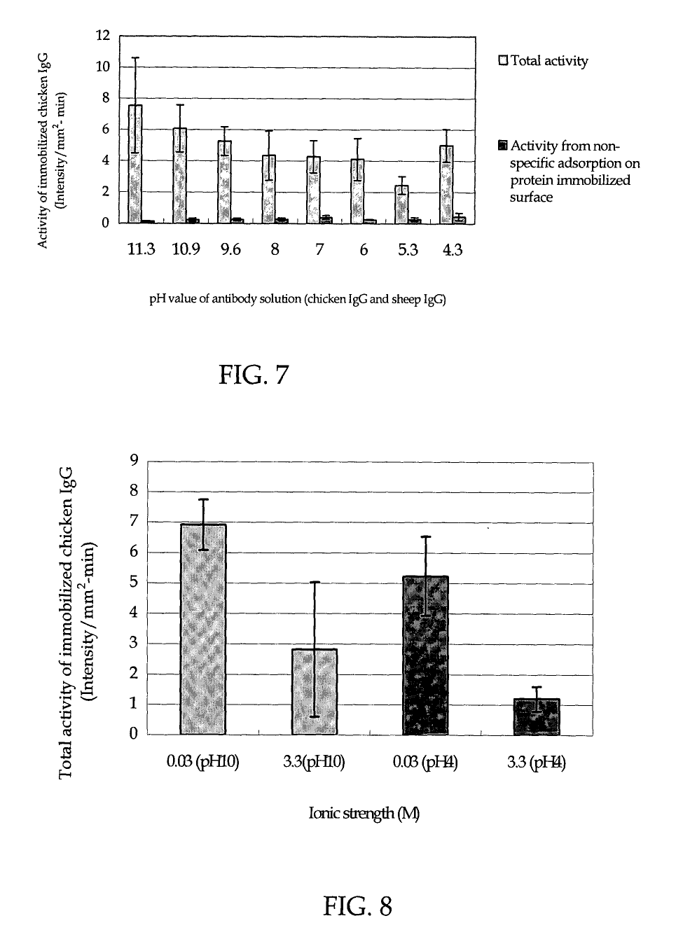

FIG. 7 shows both total and non-specific surface activities for different pH levels. When PDMS slides were incubated with chicken IgG solution at pH 11.3, the resulting total surface activity reached the maximum value, 7.5 intensity/mm²-min. The minimum surface activity, 2.4 Int/mm²-min, was found at pH 5.3. The resulting surface activity is very pH dependent where higher surface activities were found at the outer pH values of the experimental range. IgG molecules have the isoelectric points ranging from 5.3 to 7.5. The change of surrounding pH values can cause the change of IgG molecule net charge. The PDMS surface properties might change, too. It was reported that PDMS backbone is partially polar or ionic and partially covalent; therefore, PDMS is susceptible to hydrolysis at extremes of pH. Both change of chicken IgG net charge and change of PDMS surface property at different pH controls can explain the pH dependent adsorption.

The surface activities caused by non-specifically adsorbed anti-chicken IgG-P on sheep IgG attached PDMS surfaces are not significant at all pH levels. This result indicates that blocking non-specific binding sites by BSA is effective.

Influence of Ionic Strength on Surface Activity

Chicken IgG solutions with different ionic strengths were used here to incubate with PDMS slides. Whether different ionic strengths can affect chicken IgG immobilization on PDMS was investigated in this experiment. The resulting PDMS with adsorbed chicken IgG were tested for their surface activity to find a best ionic strength control for the optimization of chicken IgG adsorption.

Methods

The following buffers were prepared. The ionic strength of each buffer was calculated and controlled by adding sodium chloride and diluting with distill water.

1. Acetate buffer, pH 4, ionic strength 3.3 M

2. Acetate buffer, pH 4, ionic strength 0.03M

3. Carbonate-bicarbonate buffer, pH 10, ionic strength 3.3 M

4. Carbonate-bicarbonate buffer, pH 10, ionic strength 0.03 M

Each buffer was used to prepare a 10 μg/ml chicken IgG solution. PDMS slides were then incubated in chicken IgG containing buffers according to procedures described herein. The blocking procedures and surface activity procedures were performed as described herein.

Result

FIG. 8 shows the resulting immobilized chicken IgG activities on PDMS when incubated at two different ionic strengths and two different pH values. At either pH 10 and 4, high ionic strength resulted in less surface activity than low ionic strength. This indicates that less chicken IgG were adsorbed on the PDMS surface when the aqueous solution surrounding the substrate is at high ionic strength. It was reported that the high ionic strength reduces the protein adsorption which is by electrostatic interaction. In contrast, for the case of protein adsorption by hydrophobic interaction, the high ionic strength does not reduce the adsorption for protein adsorption on solid surfaces. Based on the report above and the results from the experiment that showed the reduced amount of adsorbed chicken IgG at higher ionic strength, the dominant interaction between chicken IgG and PDMS surface is most likely electrostatic force.

Investigation of Antibody-PDMS-Surface Interactions: Covalent Attachment

The adsorption method for chicken IgG immobilization was investigated and discussed in the previous section. In this section, the covalent binding method for chicken IgG immobilization was examined and compared with the adsorption method to determine a better method for the optimization of chicken IgG immobilization. To introduce covalent binding between antibodies and PDMS surface, the surface modification method, including silanization and crosslinking, for covalent immobilization of protein on silicon/glass surface was adapted for PDMS surface.

As stated previously, the hydroxyl group on acid-treated silicon/glass surfaces plays an essential role which allows the surface modification to proceed and thus the protein can be successfully attached on silicon/glass surfaces by covalent means. The original PDMS surface does not have any hydroxyl groups. Therefore, a novel process was studied and developed to introduce hydroxyl groups on PDMS and to adapt the reported modification methods of silicon/glass to PDMS substrates. Plasma discharge was used to change the hydrophobic property of PDMS to hydrophilic. Hydroxyl groups were found to be introduced on plasma-treated PDMS surfaces. Integrating the silicon/glass surface modification method and the plasma treatment for PDMS, a novel surface modification process, referred to herein as the three-step covalent binding method, employing three steps, plasma treatment, silanization, and crosslinking, for covalent immobilization of proteins was developed and analyzed. The attached chicken IgG by this novel immobilization process were tested for their activity, attachment saturation point, long-term stability, and antigen-capture sensitivity.

Activities of Immobilized Chicken IgG on Completely Modified, Incompletely Modified, and Non-Modified PDMS Surfaces

The purpose of this experiment is to compare the activities of chicken IgG immobilized on three different PDMS surfaces. The first PDMS surface was completely modified using the novel three-step covalent binding method, and the chicken IgG attachment was by covalent means. The second PDMS surface was incompletely modified by the first two steps of the three-step covalent binding method, including plasma and silanization treatments, and the chicken IgG was attached by adsorption. The third surface was the original non-modified PDMS and chicken IgG was immobilized by adsorption.

Methods

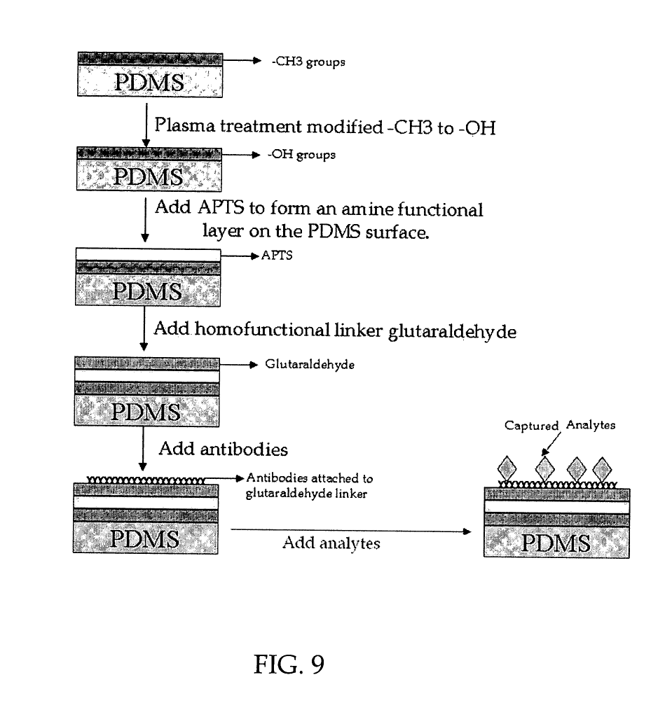

One set of PDMS elastomer slides went through the plasma treatment, silanization treatment, and crosslinking treatment for completed three-step covalent binding modification (herein referred to as completely modified); one set was subjected to plasma treatment and silanization treatment (herein referred to as incompletely modified); and one set was not treated (herein referred to as non-modified). The three-step covalent binding method consisting of plasma treatment, silanization treatment, and crosslinking treatment is illustrated schematically in FIG. 9 and described below.

a. Plasma Treatment

PDMS slides in 5 mm×5 mm×2 mm dimension were thoroughly rinsed with ethanol and dried under a stream of air. PDMS slides were placed in a plasma cleaner (PDC-001, Harrick, Ossining, N.Y.) and oxidized for 1 minute.

b. Silanization Treatment

Immediately after removal from the plasma cleaner, PDMS slides were soaked in 10:1 H₂O: aminopropyl triethoxysilane (APTS, Sigma, St. Louis, Mo.) adjusted to pH 7.0 with 10% acetic acid at 80° C. for 3 hours. Slides were then rinsed with distilled water using wash bottle for 15 seconds each side.

c. Crosslinking Treatment

PDMS slides were soaked in 10% glutaraldehyde (G6257, Sigma, St. Louis, Mo.) at room temperature for one hour. Slides were rinsed individually in a small sample vial on a vortexer to insure removal of excess glutaraldehyde and avoid crosslinking of immobilized antibody.

d. Chicken IgG Immobilization on Three Different PDMS Surfaces and Surface Activity Measurement

Three sets of PDMS slides (non-modified; incompletely modified; and completely modified) were separately incubated in PBS/pH7.4 solution containing 10 μg/ml chicken IgG in Microfuge tubes at 37° C. for one hour. The chicken IgG/PBS solution volume for incubation was 1 ml per slide. The non-specific sites blocking and the measurement of activity of immobilized chicken IgG were performed as the procedures described above. The control experiment for activity from nonspecific adsorption of anti-chicken IgG-P was also performed as described previously. These steps were also illustrated in FIG. 9.

Results

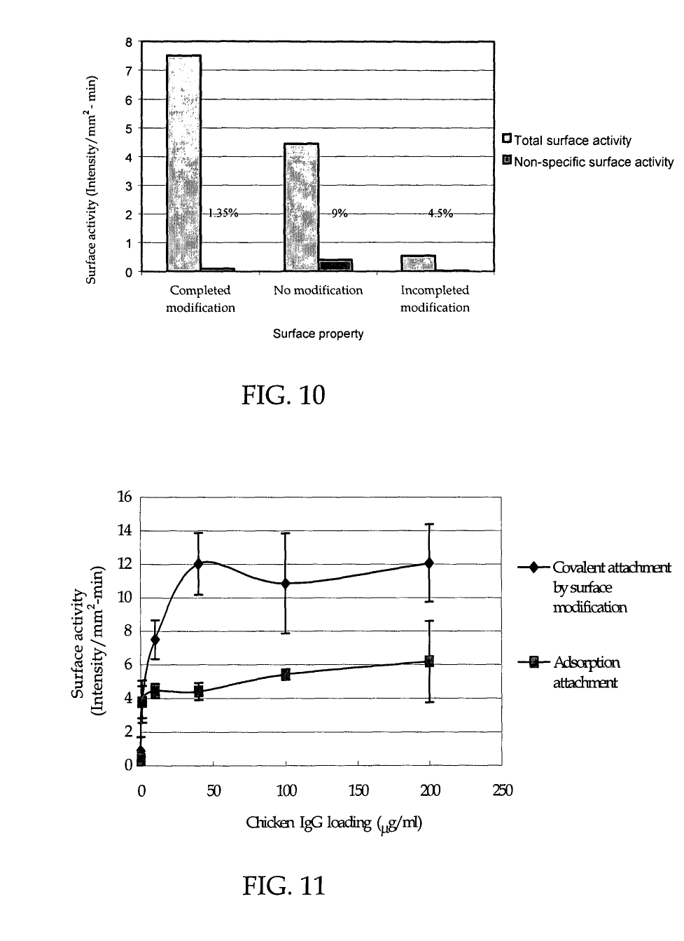

FIG. 10 shows the activities of immobilized chicken IgG by different types of attachment. When the three-step covalent binding method was fulfilled, the chicken IgG attachment by covalent binding on PDMS surface was achieved and the highest activity (7.5 Int/mm²-min) of immobilized chicken IgG was observed comparing with other samples. This activity is 1.7-fold and 15-fold of the surface activities of non-modified PDMS and incompletely modified PDMS, respectively. Therefore, chicken IgG immobilization on PDMS by the presently described novel three-step covalent binding method is a more effective method than the adsorption method in terms of the resulting surface activity. In addition, the percentage of surface activity from non-specific adsorption on completely modified PDMS surface was the least among all the surfaces, percentages shown in FIG. 10.

Investigation of the Relation between Loaded Antibody Concentration and Immobilized Antibody Amount on PDMS Surface

In this section, chicken IgG was immobilized by the three-step covalent binding method on PDMS using a series of chicken IgG loading concentrations. The resulting surface activity of chicken IgG as a function of loading IgG concentration was compared with the one obtained previously using adsorption method. The chicken IgG immobilization mechanisms by two different methods can be analyzed and compared.

Methods

PDMS slides were completely modified using the three-step covalent binding method according to procedures described above. A series of chicken IgG with six different concentrations, i.e. 0.05, 1, 10, 40, 100, 200 μg/ml, were prepared in PBS/pH7.4. Completely modified PDMS slides were incubated in 1 ml of chicken IgG solution of each concentration at 37° C. for one hour. The blocking step and surface activity measurement were performed as the procedures described previously.

Results

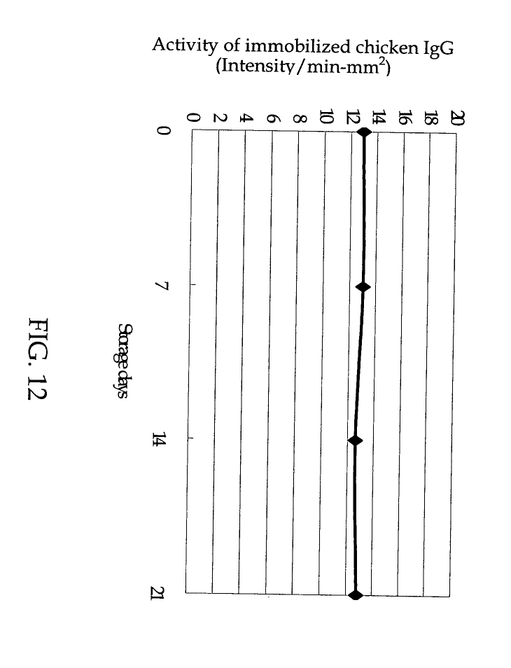

FIG. 11 shows two sets of surface activity as a function of chicken IgG concentration. Chicken IgG immobilized by surface modification using the three-step covalent binding method resulted in the higher surface activity with slower saturation rate. The saturation point is around 12 Intensity/mm²-min when chicken IgG loading concentration reaches 50 μg/ml. The saturation points can represent the maximum surface activities. Comparing the saturation points between the three-step covalent binding method and adsorption method, the maximum surface activity from the three-step covalent binding method is 2.2 times of that from adsorption attachment. The novel three-step covalent binding method was proven to be the more effective way to immobilize chicken IgG on PDMS for optimization of antigen-capturing activity.

Stability of Immobilized Antibodies on Modified PDMS Surfaces

For sensor application, the stability of immobilized affinity ligands in an affinity system is very important in terms of the sensitivity of the sensor. Therefore, the stability of the immobilized antibodies on completely modified PDMS surfaces was investigated here. One possible factor that can affect the stability of the immobilized antibodies on completely modified PDMS surfaces is hydrophobic recovery phenomenon. The hydrophobic recovery phenomenon was discussed previously. The PDMS surface was reported to be oxidized by plasma discharge to form a hydrophilic layer containing SiOx, hydroxyl, and carbonyl groups. However, cracking of this layer promotes transport of low molar mass molecules gradually to the surface, covering the oxidized hydrophilic layer, and causes the hydrophobic recovery. The major purpose of this experiment is to check whether the hydrophobic recovery phenomena of plasma treated PDMS will affect the amount and activity of covalent-attached antibodies on completely modified PDMS surfaces.

Methods

Four sets of PDMS elastomer slides were completely modified using the three-step covalent binding method. Each set was attached with chicken IgG and blocked with BSA according to procedures set forth above. The IgG attached slides were stored separately in tubes with 0.01% sodium azide-PBS (pH7.4) at 4° C. The immobilized chicken IgG activities of Set 1 to Set 4 PDMS slides were tested after four different storage durations using anti-chicken IgG-peroxidase and its substrate. The storage duration before activity test for Set1 to Set4 of PDMS slides was 0, 7, 14 and 21 days respectively. Each set contains 3 duplicates.

Results and Discussion

The resulting activities of covalent-immobilized chicken IgG on PDMS slides over 0 to 21 days of storage are illustrated in FIG. 12. The resulting activities of immobilized chicken IgG of four different storage terms showed high stability with no significant decline. The small random variants of activities (less than 3%) between each set of different storage terms were mostly caused by expected experimental control difference when performing the immunoassay at different times. Those experimental-control differences include temperature variant, pipette accuracy, antibody, enzyme, and substrate qualities in each batch when performing the immunoassay.

The high activity stability of immobilized chicken IgG on completely modified PDMS slides indicates that the amount, functionality, and immobilization status of IgG did not change by the fact of PDMS hydrophobic recovery during 21-day of storage. It shows the possibility that the PDMS hydrophobic recovery is prevented or retarded by post-plasma-treatment surface modification. The hypothesis of hydrophobic recovery prevention/retardation mechanism is proposed as following. The low molar mass molecules in PDMS will usually migrate through the cracks to the surface and gradually cover the oxidized hydrophilic layer. In this experiment, the oxidized hydrophilic PDMS surface was modified with APTS immediately and glutaraldehyde subsequently. Both of these two substances can polymerize and form multilayers of polymerization and the antibodies are linked covalently to these polymerized layers. These polymerized layers can possibly cover and block the cracks caused by the plasma oxidization and hence prevent or retard the migration of low molar mass molecules through the cracks. Therefore, the hydrophobic recovery is prevented/retarded, which might otherwise cause the changes of binding and activity of antibodies on the surfaces.

To understand the surface chemistry mechanism during the PDMS storage time, further examination by XPS and ellipsometry is needed, which allows for the investigation of the surface species composition. Even though the immobilized IgG activities test herein did not show the exact surface chemistry involved, it did reveal that the immobilized antibodies by the novel three-step covalent binding method are stable and are not affected by the hydrophobic recovery over a 3-week period.

Construction of Microfluidic Affinity System

So far, all the experiments are done on the flat PDMS slides to investigate the protein/PDMS surface interactions. Both adsorption and covalent protein/PDMS interactions were investigated and compared by the test of surface activities, or immobilized antibody activities. It is shown that the novel three-step covalent binding method for the covalent immobilization of antibodies on PDMS surfaces resulted in higher antibody loading and higher antibody activity. The covalently attached antibodies also showed high stability in a 21-day storage term. With the improved performance and stability results, the novel three-step covalent binding method was chosen to be used for the construction of a microfluidic affinity system.

Fabrication of Fluidic Channels on PDMS Elastomer and the Enclosure of Channels

The first step for the construction of the microfluidic affinity system is to fabricate a fluidic channel pattern on a PDMS elastomer. The fabrication methods are based on the replication using patterned silicon wafer as the mold. One PDMS cast with fluidic channel pattern and one PDMS flat slab were sealed to each other by the silanol condensation reaction. This condensation reaction was discussed previously, whereby plasma discharge converts —OSi(CH₃)₂O— groups at PDMS surfaces to —OₙSi(OH)₄ₙ—. When two oxidized PDMS were brought to conformal contact, condensation reaction between two silanol groups on two contact surfaces results in covalent siloxane bonds, Si—O—Si. Reversible binding between two oxidized PDMS elastomers is expected in this experiment to form the enclosed fluidic channels. To test if this sealing method is applicable for the microfluidic affinity system, fluorescent dye is pumped through the sealed fluidic channels and are observed under a fluorescent microscope.

Methods

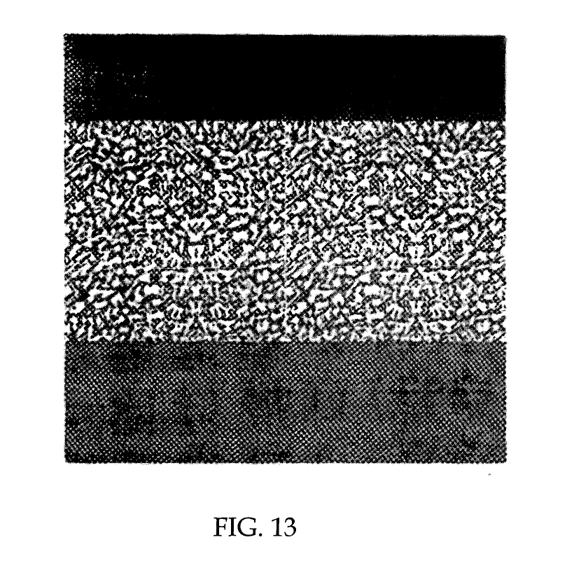

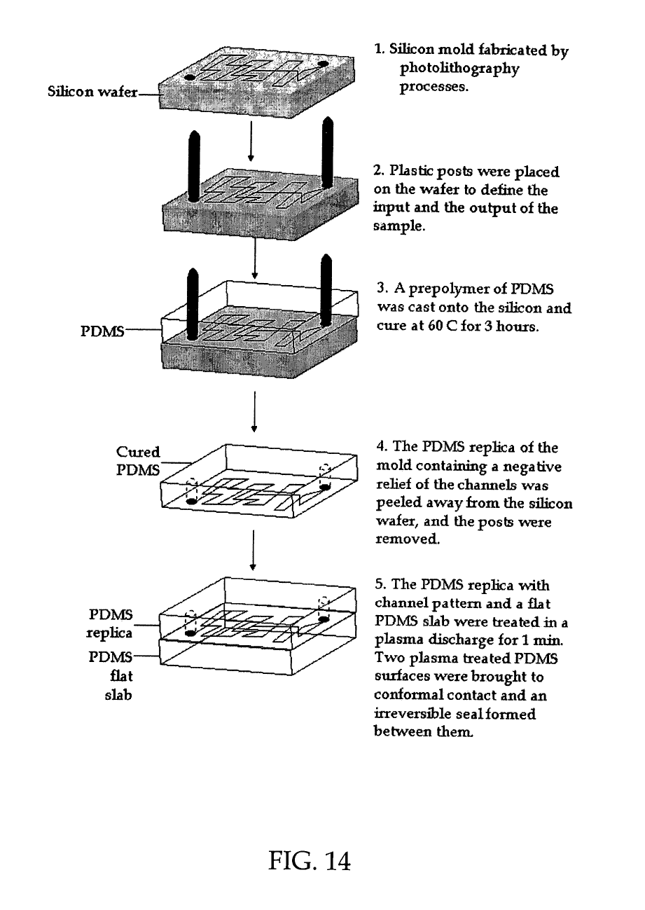

A standard silicon-based lithographic process was used to controllably generate a master pattern onto a silicon wafer. This master wafer, with reversed fluidic channel patterns, was then used repeatedly as a mold for PDMS cast for pattern transfer to the PDMS substrate. The reversed channel pattern was designed by the Computer Aided Designed (CAD) program SYMBAD to simulate porous media, see FIG. 13. The inverted microfluidic pattern on silicon master was 20 μm in depth and 20 to 70 μm in width. Two plastic posts were placed on the input and output locations on the silicon master. Then, PDMS liquid was poured on this silicon master with two posts, cured, cut, and pealed off from the mold as the procedures described previously. Another flat PDMS slab was made the same way but using a flat bottom container as the mold. Two PDMS elastomers, one with patterns and one without patterns, were treated with plasma cleaner (PDC-001, Harrick, Ossining, N.Y.) for 1 minute. Two plasma-treated PDMS elastomers were immediately brought together to make a conformal contact; seal can form immediately. The procedures are illustrated schematically in FIG. 14.

To test the reliability of this sealing method, the resulting enclosed fluidic channel in PDMS elastomers was connected with two plastic tubes on the input and output locations. One tube was linked between a syringe (5 ml) containing fluorescent dye (Rhodamine chloride 560, Exciton) and the input; the other tube was linked between the output and an effluent collection container. The syringe containing fluorescent dye was pumped by a syringe pump (Kd Scientific) at the controlled flow rate. Four flow rates were tested: 1, 2, 4, 6 ml/hr. The flow conditions were observed under a fluorescent microscope (Wild M3Z, Leca) to check if there was any leaking condition.

Results

The conformal contact between two plasma-treated PDMS elastomers, one PDMS replica with a network of fluidic channels and one flat slab of PDMS, resulted in tight binding between them. This seal between the two pieces of PDMS was sufficiently strong that two substrates could not be peeled apart without failure in cohesion of the bulk PDMS. However, this successful sealing can be achieved only when the surfaces of two pieces of PDMS used for plasma treatment were very clean without any contamination from dust in the air or the grease on hands. Hence, two pieces of PDMS used in this experiment for sealing processes were both new-cured PDMS without exposing to air for long.

All PDMS pieces, even those cleaned with ethanol, not newly made resulted in sealing failure due to the contamination on the surfaces. The contamination on the PDMS surfaces can interfere with the access of plasma discharge to PDMS surface groups, thus the PDMS surfaces can not be oxidized well to form enough silanol groups. Without sufficient silanol groups on PDMS surfaces, the formation of covalent siloxane bonds by the condensation reactions between silanol groups on two PDMS substrates can not be achieved. This is the most likely explanation for the failure of sealing between not newly made PDMS substrates.

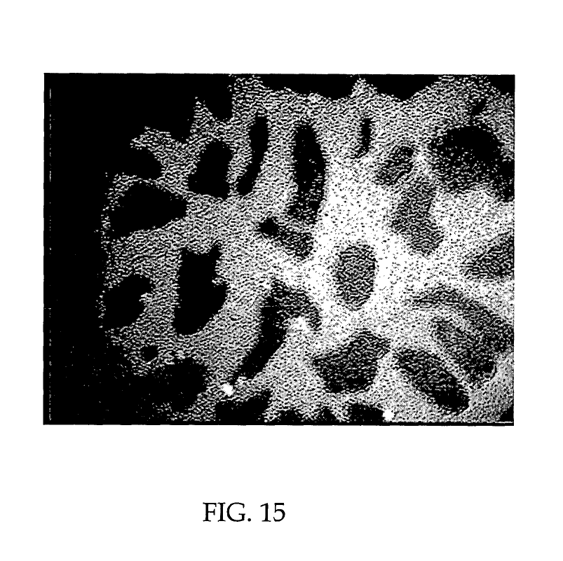

Newly-cured PDMS substrates result in good sealing after oxidization and conformal contact. The fluorescent dye pumped through the sealed fluidic channels at 1, 2, 4, and 6 ml/hr all flowed well in the defined areas when observed under a fluorescent microscope. The leaking and flowing out of defined areas were not found in this experiment. The flow condition picture taken from the CCD camera connected to fluorescent microscope is shown in FIG. 15. This experiment showed that the sealing method by plasma treatment and PDMS conformal contact is an effective way for enclosed fluidic channel construction.

Modification of PDMS Fluidic Channel Wall, Immobilization of Chicken IgG, and Separation System Efficiency Test

Covalent immobilization of IgG on PDMS surfaces by the three-step covalent binding method was examined in the previous experiments and resulted in higher IgG loading and higher activity to capture its antigen comparing with the adsorption attachment of IgG. Therefore, the covalent immobilization method was chosen to be used in the microfluidic affinity system having PDMS as the substrate.

However, the experimental conditions in previous flat protein-surface interaction and the microfluidic affinity system are different. For example, the previous protein-surface interaction experiments were done on flat PDMS slides and were in a batch environment; and the present experiment was done on channel walls and was in a continuous flow environment. Whether the chicken IgG can be immobilized successfully and retain their activity to capture its antigen in a microfluidic affinity system was investigated in this experiment section. The antigen capture activity of immobilized antibodies in PDMS fluidic channels was analyzed by the comparison of the amount of analytes, such as anti-chicken IgG antibody-peroxidase, in input samples and output samples. This analyte-capturing activity also represents the separation efficiency of the microfluidic affinity system. Therefore, this experiment is essential in determining the success of the system developed herein.

Methods

Surface Modification

Two plasma-treated PDMS elastomers, one PDMS replica with a network of fluidic channels and one flat slab of PDMS, were brought together to form a seal as described above. Right after the sealing step, the enclosed fluidic channels were immediately injected with 10% APTS at 6 ml/hr using syringe and pump for 5 minutes to make sure that APTS covers the whole flow area in the fluidic channels. The pump was then stopped and the fluidic channels filled with APTS were placed in an 80° C. oven for 3 hours. Fluidic channels were rinsed with 5 ml distill water at 6 ml/hr using syringe pump. 10% glutaraldehyde was injected into the fluidic channels using a syringe pump for 5 minutes to make sure that glutaraldehyde covers the whole flow area. The pump was then stopped and the fluidic channels filled with glutaraldehyde were left standing for 1 hour at room temperature. Fluidic channels were rinsed with 5 ml distill water at 6 ml/hr using a syringe pump. Two sets of microfluid affinity systems were prepared according the procedures above.

IgG Immobilization and BSA Blocking

Both sets of microfluidic affinity systems were manipulated as follows. 50 μg/ml Chicken IgG in PBS/pH 7.4 was pumped to the fluidic channel at 6 ml/hr for 5 minutes. Pumping was stopped to let the fluidic channels filled with chicken IgG solution stand for one hour at 37° C. The fluidic channels were rinsed with 5 ml of PBS/pH7.4/0.05% Tween 20 at 6 ml/hr at room temperature. 1% BSA in PBS/pH 7.4 was pumped to fluidic channels at 6 ml/hr for 5 minutes and the fluidic channels with BSA solution was incubated at 37° C. for 1 hour. Fluidic channels were rinsed as described above.

Analyte-Capturing Test

Two different concentrations of anti-chicken IgG antibody-peroxidase were prepared for two microfluidic affinity systems. They were 10 ml of 1.3 μg/ml and 0.47 μg/ml anti-chicken IgG antibody-peroxidase in PBS/pH 7.4 solutions. 1 ml of each solution was taken out and stored on ice as the input reference. The rest of the 9 ml of each anti-chicken IgG antibody-peroxidase solution was placed in a glass syringe and pumped into the fluidic channel using a syringe pump at the flow rate of 6 ml/hr. The syringe was covered with ice bag. Only the first 5 fractions were collected. Each fraction was 0.5 ml in volume, collected at a 5-minute interval, and stored on ice immediately. The rest of the 6.5 ml input solution was continuously pumped through the fluidic channels and the last 0.5 ml of output was collected to check the saturation status. The first 5 fractions and 1 last fraction of output samples and one input reference sample from each microfluidic affinity system were obtained. 50 μl of each sample was taken out and added to 500 ml of peroxidase substrate (SuperSigal ELISA Pico Chemiluminescent Substrate, Pierce, Rockford, Ill.) and the chemiluminescent intensity was measured with a spectrophotometer. The standard curve of anti-chicken IgG-P versus intensity was constructed based on the previously obtained data. This standard curve was used to convert the measured intensities to corresponding anti-chicken IgG-peroxidase concentrations. The calculation for the total amount of input anti-chicken IgG-peroxidase and total amount of captured anti-chicken IgG-peroxidase are in the following.

Total amount of input=(input concentration)×(total volume of input)

Total amount of capture=(Total amount of input)−Σ(0.5 ml)(output concentration of each fraction)

Results

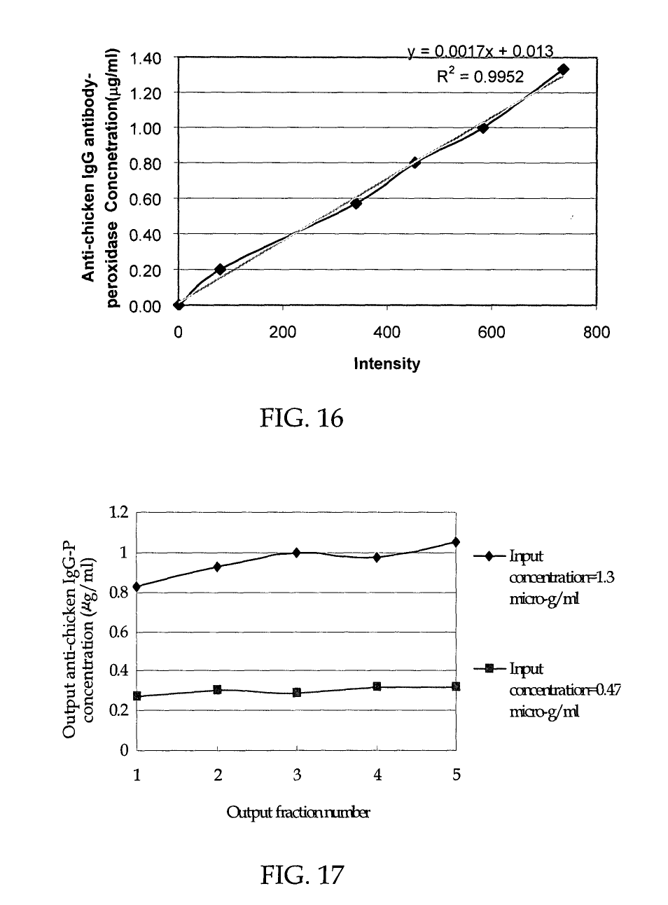

The standard curve for the relation between anti-chicken IgG-P and intensity is shown in FIG. 16. The equation for this standard curve was obtained to covert the measured intensity to the corresponding anti-chicken IgG-P in this experiment.

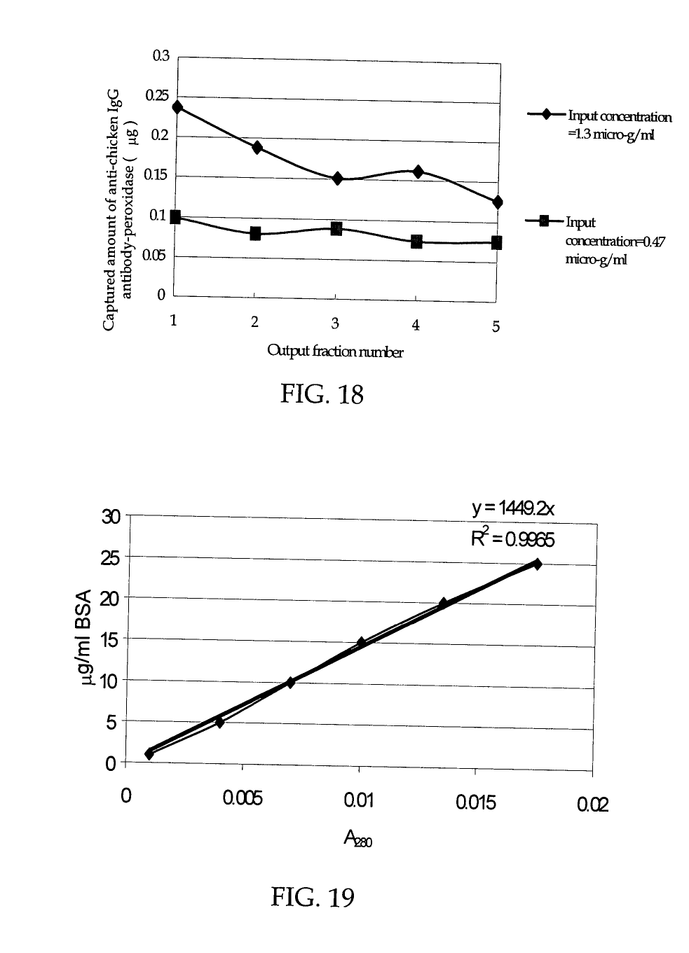

FIG. 17 shows the resulting output anti-chicken IgG-peroxidase, or analyte, concentrations when two different input concentrations were applied. For both input conditions, the output analyte concentrations are significantly different from that of input. This indicates that the immobilized chicken IgG in the fluidic channels retained their activity and would be able to capture the analytes from the input samples. For the higher analyte concentration input, 1.3 mg/ml, the output analyte concentration show a trend of increase; for the lower analyte concentration input, 0.47 mg/ml, the output analyte concentration shows less increase. The increasing output analyte concentration IgG in the fluidic channels are going to be saturated as sufficient amount of analytes are applied and captured. This explains how the higher analyte concentration input resulted in faster saturation trend then the lower analyte concentration input. FIG. 18 shows the removed, or captured by immobilized chicken IgG, amount of analytes from each output fraction for both high and low input concentrations. This result more directly shows how a higher concentration input caused a higher amount of analyte capturing and resulted in faster chicken IgG binding site saturation.

Both fluidic channel systems with 1.3 and 0.47 mg/ml analyte input concentrations were saturated after 9 ml of sample ran through. This is based on the result that the output sample had the same analyte concentration as the input. Total amounts of captured analytes over 5 fraction collection for each input analyte concentrations were summarized in the following Table 1.

TABLE 1

| | System 1 (higher | System 2 (lower input |
|---|---|---|
| Microfluidic affinity system | input concentration) | concentration) |
| Input analyte concentration | 1.3 | 0.47 |
| (μg/ml) | | |
| Total amount of input | 3.25 | 1.16 |
| analytes (μg) | | |
| Total μg of captured | 0.86 | 0.42 |
| analytes in 5 fractions | | |

Table 1 shows that the total amounts of captured analytes, such as anti-chicken IgG antibody-peroxidase, are nearly proportional to the input analyte concentrations. When the input analyte concentration increased 2.7 fold, the amounts of captured analytes increased 2.1 fold. This result reveals the applicability of this microfluidic affinity system for sensor application that the total captured amounts of analytes are proportional to the total input analytes.

Release Captured Analytes from Antibody

In previous experiments, the microfluidic affinity system was shown to have the ability to sort the analytes from input samples by antibody-antigen interactions. Therefore, the recognition and separation of analytes from solution by the novel microfluidic affinity system have been achieved. In order to adapt this microfluidic affinity system to a previous studied electronic measurement system, the sorted analytes in the microfluidic affinity system need to be released and sent to the electronic measurement system. The main purpose of this experiment is to investigate the effectiveness of analyte releasing methods and to analyze the reusability of this microfluidic affinity system.

Methods

Standard Curve Construction

The standard curve for protein concentrations versus their corresponding adsorption at 280 nm wavelength, A₂₈₀, was constructed by measuring the A₂₈₀ of BSA solutions (1 to 25 μg/ml). The equation for the relationship of A₂₈₀ and protein concentrations can be generated.

Analytes Release

The saturated microfluidic affinity system which was previously applied with 0.47 μg/ml of anti-chicken IgG-peroxidase input was rinsed with 5 ml of glycine-HCl (pH 2.3) at 6 ml/hr at room temperature. The output fractions were collected. Each was in the volume around 1.5 ml. Output fractions and one sample of glycine-HCl buffer, as a blank reference, without running through the fluidic channels were measured for the adsorption at 280 nm using the adsorption spectrophotometer. The analyte concentrations corresponding to measured A₂₈₀ were calculated using the standard curve obtained above. The fluidic channels were neutralized by rinsing with 6 ml PBS/pH7.4 at 6 ml/hr at 4° C.

Reuse of Microfluidic Affinity System

2.5 ml, 0.5 μg/ml of anti-chicken IgG-peroxidase solution was injected into the regenerated fluidic channels. The output fractions were collected and compounds were added for analyzing the chemiluminescent intensity. The total input analytes amount and total captured antigen from 5 output fractions were also calculated. The fluidic channels were then regenerated again using glycine-HCl (pH2.3) as described above. The resulting regenerated fluidic channels were tested with analyte containing sample (0.58 μg/ml) and the output fractions were analyzed following the same procedures before.

Results

The standard curve for the protein concentrations and their corresponding absorption at wavelength of 280 nm was constructed as shown in FIG. 19. The equation of protein concentration as a function of A₂₈₀ was also shown in FIG. 19. The reason for using A₂₈₀ to analyze the released analytes, or anti-chicken IgG-peroxidase, concentration, instead of using chemiluminescent intensity from peroxidase-substrate reaction, is that the solution used to cause analytes release from chicken IgG is very acidic, glycine-HCl/pH2.3, and it might affect the peroxidase conjugated on antigen activity. Hence, the assay based on peroxidase-substrate reaction is not suitable here to reflect the real released analytes concentration.

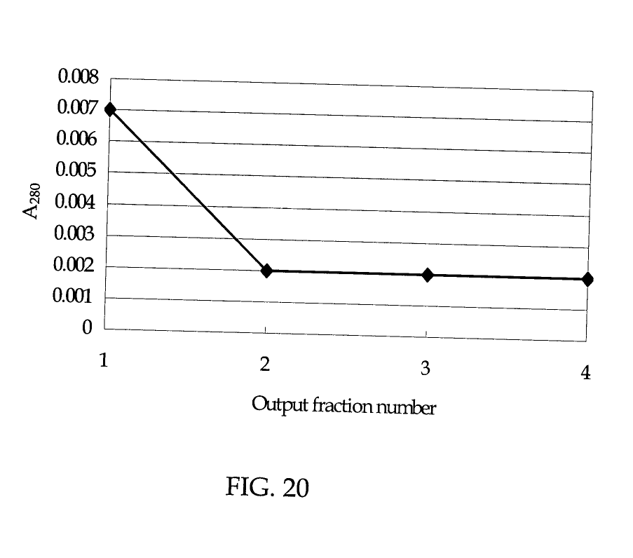

The A₂₈₀ of each output fraction when rinsing the channels with glycine-HCl solution is shown in FIG. 20. The A₂₈₀ peak (A_{280=0.007}) appears at the first output fraction and indicates that proteins were released in the first fraction. A₂₈₀ values of number 2-4 fractions are all 0.002 which is the same as that of the blank reference sample (A₂₈₀=0.002). This indicates the absence of released proteins in number 2-4 fractions. The concentration and total amount of released proteins in #1 fraction was demonstrated in Table 2.

TABLE 2

| | | Released protein | Total released |
|---|---|---|---|
| Fraction number | A280 | (μg/ml) | amount (μg) |
| 1 | 0.007 | 7.25 | 13.04 |

It is found that the released protein amount, 13.04 mg, is much higher than the total input analyte amount, 4.18 mg. As a result, the total released proteins from the fluidic channels by low pH solution rinsing could be a mixture of released analytes, chicken IgG, and BSA used for non-specific blocking. One thing that should be mentioned is that methods used to obtain the released protein amount and the total captured analyte amount are different, A₂₈₀ measurement and chemiluminenscent intensity respectively. Therefore, the released protein amount and the total captured analyte amount might not be comparable.

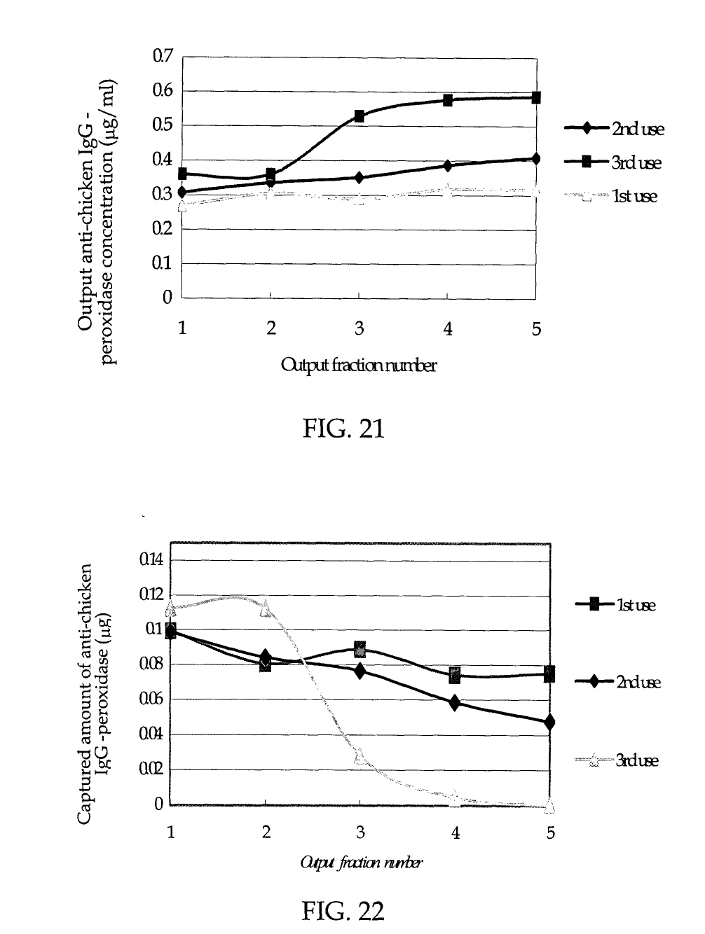

The low pH treated microfluidic affinity system above was checked for its reusability. The system was used a total of three times with acidic solution treatment using glycine-HCl, pH2.3 applied two times. The resulting performance of each of the three usages of the microfluidic affinity system is illustrated in FIGS. 21 and 22. The total captured analyte amount and the percentage of captured analytes over total input analytes are summarized in Table 3. The second use resulted in the analyte-capturing activity that is close to the activity of the first use. This means that most anti-chicken IgG-peroxidase, or antigen, were released and most of the immobilized chicken IgG in the fluidic channels were not removed and still retained their activity to capture analytes after the first acidic-rinsing.

TABLE 3

| Usage time | 1st | 2nd | 3rd |
|---|---|---|---|
| Input analyte concentration (μg/ml) | 0.47 | 0.50 | 0.59 |
| Total input analyte amount (μg) | 1.16 | 1.26 | 1.46 |
| Total captured analytes (μg) | 0.42 | 0.37 | 0.26 |
| Percentage of captured analytes over total | 35.8% | 29.0% | 17.6% |
| input analytes | | | |

This result also shows that the majority of released protein types during the first acidic rinsing experiment above are analytes and BSA. However, the small decrease of analyte-capturing activity at the second use still shows the possibility of chicken IgG detachment from the fluidic channels and protein denature while rinsing with glycine-HCl.

The analyte-capturing activity of the third use showed a very significant decrease compared with the first two uses. This might be caused by the chicken IgG detachment and protein denature from two acidic rinsing and longer storage time. From all the data obtained from analyte releasing and fluidic channel reuse experiments, two things are explored. First, captured anti-chicken IgG-peroxidase molecules can be released from chicken IgG immobilized in the fluidic channels by rinsing with glycine-HCl (pH 2.3). It is believed that the low pH glycine-HCL can cause changes of protein folding and hence cause changes of the protein conformation. The antibody-analyte interactions are conformation depending, so analytes can be released from antibody when the conformations of them are changed by low pH glycine-HCl.

Second, it was observed that BSA and chicken IgG molecules might be rinsed off from the fluidic channels and the immobilized chicken IgG might lose their activity especially when treated with a second glycine-HCl rinsing. Therefore, this microfluidic affinity system is not suitable for reusing more than twice and the analyte releasing method needs to be further improved for the integration with the electronic measurement system.

Testing Microfluidic Affinity System with Cryptosporidium parvum Oocysts

The microfluidic affinity system was tested with anti-chicken IgG-peroxidase molecules as the analytes in previous experiments. In this experiment, a pathogen, Cryptosporidiun parvum oocyst, will be separated as the analytes from input solution by the microfluidic affinity system. Previous experiments show that this microfluidic affinity system was able to capture as high as 36% of anti-chicken IgG molecules from input solution. However, cryptosporidium oocyst, which is micrometer-scale, is much larger in size then anti-chicken IgG-peroxidase molecule, which is nanometer-scale. Whether the size of the analyte will constrain the separation performance of the microfluidic affinity system is an important point for investigation here. The effectiveness of oocyst releasing from the fluidic channels by glycine-HCl (pH 2.3) solution rinsing was also investigated here.

Methods

A modified microfluidic affinity system was immobilized with 50 mg/ml anti-cryptosporidium oocyst IgM according to previously described procedures. 2 ml of PBS/pH7.4 solution containing 10⁴ cell/ml Cryptosporidium parvum oocysts was prepared. 1 ml of it was taken out and stored at 4° C. as the input reference. The rest of the 1 ml analyte solution was placed in a glass syringe and injected into the fluidic channels using a syringe pump at 1 ml/hr flow rate. The output solution was gathered and stored at 4° C. The fluidic channels were then rinsed with 5 ml of glycine-HCl (pH 2.3) at 6 ml/hr to release the captured oocysts from immobilized IgM in the fluidic channels. The output glycine-HCl sample was collected. 1 ml of output sample, 1 ml of input reference sample, and 5 ml glycine-HCl output were separately filtered with filter membranes (7060-1308, Whatman. Clifton, N.J.) using SH13 syringe filter holder (1980-001, Whatman. Clifton, N.J.). The resulting filter membranes from input reference and output sample were observed under the microscope at 100×, oil immerse (Eclipse E400, Nikon). The numbers of oocysts under 5 different microscope fields on each membrane were counted and recorded.

Results

The analyte samples used in this experiment only contained one microbe species, which is Cryptosporidium parvum oocyst. Therefore, it was possible to recognize the morphology and to count the number of Cryptosporidium oocysts under a microscope. The counted oocyst numbers at 5 different microscope fields for each sample were averaged and illustrated in Table 4. The oocyst numbers here do not represent the total oocyst number in filtered sample solutions. The number illustrated here only represents the averaged oocyst amount in the area of one microscope field size. However, these numbers are very useful to analyze relevant comparisons between each sample.

TABLE 4

| | | | Glycine-HCl Output |
|---|---|---|---|
| | | | (released oocyst |
| Sample Source | Input Reference | Output | number |
| Average Oocyst | 4.2 | 0 | 0.2 |
| Number | | | |

Zero oocysts were found in the output sample and it indicated that near 100% of oocysts in the input sample were captured by anti-cryptosporidium IgM immobilized on the walls of the fluidic channel. Only 0.2 oocyst was found on glycine-HCl output filter membrane. Therefore, a very small amount of captured oocysts was released by glycine-HCl rinsing. When we assume 100% of input oocysts were captured and use 4.2 and 0.2 as index number as captured oocyst amount and released oocyst amount respectively, the percentage of released oocysts over the total input oocysts can be obtained as 4.7%. The glycine-HCl solution with pH 2.3 was shown not effective to release oocysts from IgM.

In this experiment, a large size analyte showed no constraint to the separation performance by the microfluidic affinity system and the resulting analyte separation was quite effective. The higher effective analyte capturing here might contribute to the multivalent property of IgM used as the immobilized affinity ligand, compared with the previous experiment using IgG as the affinity ligand. The interactions between IgM and captured oocysts are probably stronger than that between IgG and anti-chicken IgG-peroxidase molecules. As the result, the glycine-HCl rinsing for releasing oocysts from IgM is not as effective as for releasing anti-chicken IgG-peroxidase from chicken IgG. The suggestion for the improvement of Cryptosporidium releasing rate is to apply lower pH solution with higher flow rates to help Cryptosporidium denature and to increase the shear force for Cryptosporidium detachment.

Although the present invention has been disclosed in terms of a preferred embodiment, it will be understood that numerous additional modifications and variations could be made thereto without departing from the scope of the invention as defined by the following claims:

We claim:

1. A method for immobilizing an affinity ligand onto a substrate comprising,
a) subjecting said substrate to a plasma treatment;
b) subjecting said substrate to a silanization treatment; and
c) subjecting said substrate to a crosslinking treatment.

2. The method of claim 1, whereby said substrate is a silicon substrate.

3. The method of claim 2, whereby said silicon substrate is a polydimethylsiloxane substrate.

4. The method of claim 1, whereby said step of subjecting said substrate to a silanization treatment includes subjecting said substrate to a silane selected from the group comprising aminosilane, sulfhydrylsilane, and epoxysilane.

5. The method of claim 4, whereby said silane is aminopropyltrimethoxysilane or mercaptopropyltrimethoxysilane.

6. The method of claim 1, whereby said step of subjecting said substrate to a crosslinking treatment includes subjecting said substrate to glutaraldehyde or N-γ-maleimidobutyryloxy succinimide ester.

7. The method of claim 1, whereby said substrate includes a fluidic channel.

8. The method of claim 7, whereby said fluidic channel is fabricated by silicon-based lithography.

9. The method of claim 1, further comprising,
d) binding said affinity ligand to said substrate.

10. The method of claim 9, whereby said affinity ligand is an antibody.

11. The method of claim 9, whereby said affinity ligand is an anti-cryptosporidium oocyst IgM.

12. A microfluidic affinity system comprising,
a) a substrate subjected to a plasma treatment, a silanization treatment, and a crosslinking treatment; and
b) an affinity ligand bound to said substrate;

13. The microfluidic affinity system of claim 12, whereby said substrate is a silicon substrate.

14. The microfluidic affinity system of claim 13, whereby said silicon substrate is a polydimethylsiloxane substrate.

15. The microfluidic affinity system of claim 12, whereby said silanization treatment includes subjecting said substrate to a silane selected from the group comprising aminosilane, sulfydrylsilane, and epoxysilane.

16. The microfluidic affinity system of claim 15, whereby said silane is aminopropyltrimethoxysilane or mercaptopropyltrimethoxysilane.

17. The microfluidic affinity system of claim 12, whereby said crosslinking treatment includes subjected said substrate to glutaraldehyde or N-γ-maleimidobutyryloxy succinimide ester.

18. The microfluidic affinity system of claim 12, whereby said substrate includes a fluidic channel.

19. The microfluidic affinity system of claim 12, whereby said affinity ligand is an antibody.

20. The microfluidic affinity system of claim 12, whereby said affinity ligand is an anti-cryptosporidium oocyst IgM.
